(19) United States
(12) Patent Application Publication
(10) Pub. No.: US 2014/0120162 A1
(43) Pub. Date: May 1, 2014

(54) Bioadhesive Drug Delivery Compositions

(71) Applicants: Edith Mathiowitz, Brookline, MA (US); Solomon S. Steiner, Mount Kisco, NY (US); Bryan E. Laulicht, Danbury, CT (US); Sasha Bakhru, Providence, RI (US)

(72) Inventors: Edith Mathiowitz, Brookline, MA (US); Solomon S. Steiner, Mount Kisco, NY (US); Bryan E. Laulicht, Danbury, CT (US); Sasha Bakhru, Providence, RI (US)

(73) Assignee: PEROSPHERE INC., Bedford, NY (US)

(21) Appl. No.: 14/124,040

(22) PCT Filed: Jun. 6, 2012
(86) PCT No.: PCT/US2012/041120

§ 371 Date: Dec. 5, 2013

Related U.S. Application Data
(60) Provisional application No. 61/493,837, filed on Jun. 6, 2011.

(51) Int. Cl. A61K 47/34; A61K 47/32

(52) U.S. Cl. CPC A61K 47/34; A61K 47/32
USPC 424/464; 514/56; 514/567

(57) ABSTRACT

Compositions containing one or more active agents, one or more bioadhesives elements, and one or more charge masking agents are described herein. In some embodiments, the one or more active agents are biomolecules or macromolecules, such as polysaccharides, proteins, peptides, or nucleic acids, which are charged at physiological pH. The one or more charge masking agents are selected based on the nature of the charge on the active agent. The compositions may also contain one or more controlled release materials, such as extended or sustained release materials or delayed release materials, in order to modify release of the active agent.

### CROSS-REFERENCE TO RELATED APPLICATION

This application claims priority to provisional application U.S. Ser. No. 61/493,837, filed Jun. 6, 2011, the disclosure of which is hereby incorporated in its entirety.

### FIELD OF THE INVENTION

The present invention is generally in the field of compositions for drug delivery containing an active agent, particularly a charged active agent, one or more bioadhesive elements, and one or more charge masking agents.

### BACKGROUND OF THE INVENTION

Systemic and local delivery of macromolecules, such as polysaccharides, proteins, peptides, and nucleic acids, faces several challenges. For example, the large size of these biomolecules is an impediment to intercellular (transcellular) and intracellular (paracellular) passage of these biomolecules. Moreover, these macromolecules are typically charged under physiological conditions. Charge can also be an impediment to intercellular and intracellular passage due to adverse interactions with other charged species as well as aggregation of the macromolecule itself.

There is a need for formulations with enhanced uptake and improved bioavailability of macromolecules, particularly charged macromolecules.

Therefore, it is an object of the invention to provide compositions that provide increased uptake and thus improved bioavailability, particularly for charged active agents, and methods of making and using thereof.

It is another object of the invention to provide formulations that enhance uptake and thus improve bioavailability, particularly for charged active agents.

### SUMMARY OF THE INVENTION

Formulations containing one or more active agents, one or more bioadhesive elements, and/or one or more charge masking agents are described. In some embodiments, the one or more active agents are macromolecules, such as polysaccharides, proteins, peptides, or nucleic acids, which are charged at physiological pH, wherein the charge may impact uptake, stability, aggregation, activity and/or interaction with other molecules. In other embodiments, the one or more active agents can be small molecule active agents, which are preferably charged under physiological conditions. The one or more charge masking agents are selected based on the chemical structure and the charge(s) on the active agent(s). The one or more active agents, one or more bioadhesive elements, and/or one or more charge masking elements may be formulated as nanoparticles or microparticles, the size of which will determine the uptake, the specific location of uptake, and the residence time of the active agent.

In some embodiments, the bioadhesive elements may be dispersed in the matrix of a solid oral dosage form or applied as a direct compressed coating to a solid oral dosage form. Preferred bioadhesive polymers include poly (adipic)anhydride “p(AA)” and poly (fumaric-co-sebacic) anhydride “p[FA:SA]”. Other preferred bioadhesive polymers include non-erodable polymers such as DOPA-maleic anhydride co polymer; isopthalic anhydride polymer; DOPA-methacrylate polymers; and DOPA-cellulosic based polymers. Additional polymers include polycyclic acid, EUDRAGIT®, chitosan, and zein.

The compositions may also contain one or more controlled release materials, such as extended or sustained release materials, delayed release materials, or combinations thereof, in order to modify release of the active agent. The controlled release elements are selected to determine the site of release. For example, an enteric coating can be used to delay release until the formulation reaches the ileum; additional controlled release elements can be used to further delay release so that release occurs within the first one-third of the small intestine; the second one-third; or the last one-third of the small intestine. The controlled release elements can also be selected to delay release until the drug formulation reaches the colon. The bioadhesive components are selected to provide retention of the formulation at the desired site of uptake. This will occur after the enteric coating, if present, dissolves, or immediately after administration if no coating is applied. By selecting for both release and retention at a specific site, typically based on time of transit through the gastrointestinal tract, one can obtain enhanced efficacy of uptake of the drug. The controlled release elements can be dispersed in the matrix of a solid oral dosage form or applied as a direct compressed coating to a solid oral dosage form, such as a tablet or capsule.

The formulation is typically in the form of a tablet or capsule, which may include microparticles, nanoparticles, and/or beads. The formulations use bioadhesive elements, charge masking elements, and/or controlled release elements to direct release to specific regions where the bioadhesive elements are exposed at the time the formulation reaches the region of desired release. This can result in enhanced absorption relative to the formulation in the absence of the bioadhesives, charge masking, and/or controlled release elements. This is demonstrated by several examples showing delivery of different drugs having greater area under the curve (“AUC”) relative to the reference immediate release dosage form, i.e., the AUC of the composite bioadhesive formulation is greater than 100% of the AUC of the immediate release drug and/or the drug in a formulation containing only the controlled release or bioadhesive elements. In the preferred embodiment, the area under the curve is at least 10%, 15%, 20%, 30%, 40%, 50%, 100%, or 200% of the reference formulation. The AUC is also typically greater than the AUC of non-bioadhesive controlled release formulations and bioadhesive/controlled release formulations lacking the one or more charge masking elements. In some embodiments, the reference formulation does not contain one or more charge masking elements. In other embodiments, the reference formulation does not contain one or more charge masking agents or one or more bioadhesive elements.

In one embodiment, the compositions are formulated for local administration of any heparin, such as unfractionated heparin (also referred to as heparin) or fractionated heparins, such as low molecular weight heparin (LMWH), or very low molecular weight heparin (VLMWH) to the GI tract to treat Crohn's disease, Irritable Bowel Syndrome, and/or colitis. In another embodiment, the compositions described herein are formulated for systemic administration of any heparin, such as heparin, LMWH, or VLMWH via the oral route. In this embodiment, heparin can be formulated in a bioadhesive microsphere or nanosphere containing one or more charge masking elements and encapsulated in a hard or soft capsule, such as a gelatin capsule. The capsule can be coated with a pH-sensitive polymer, such as a EUDRAGIT®. Alternatively, the capsule itself can contain an enteric polymer which avoids the need for a polymer coating. The compositions containing heparin can also be formulated as a tablet. Formulations for systemic delivery of heparin described herein may provide pain free delivery, reduce peak-to-trough effects, improve patient compliance, be used to treat larger and/or more diverse patient populations, and/or be used to treated new indications.

In other embodiments, the compositions are formulated for administration of Copaxone for the treatment of multiple sclerosis (MS). The charge masking agent can be a peptide containing the same peptides as Copaxone, wherein the ratio of lysine and glutamic acid are reversed, i.e., ˜5Ala:1.5Lys:3Glu:1Tyr. At physiological pH, Copaxone has a net positive charge with an overall hydrophobic nature. Reversing the ratios of lysine and glutamic acid increases the amount of negative charge on the masking agent which can mask the positive charge of Copaxone. Negatively charged amino acids, such as glutamic acid or aspartic acid, can also be added to mask the charge on Copaxone. Lesser amounts of positively charged amino acids can also be used to mask the negatively charged glutamic acid residues in Copaxone. In another embodiment, Copaxone can be charged masked by the bioadhesive element itself. For examples, polyanhydrides degrade to dicarboxylic acids, such as fumaric acid. These acids can mask the positively charged Copaxone.

The combination of charge masking agents with nano- and/or microencapsulation and bioadhesion can: (1) stabilize natural and synthetic polypeptides, proteins, enzymes, antibodies and polysaccharides, (collectively designated as therapeutic agents); (2) target the therapeutic agent to specific locations within the body; (3) enhance the systemic bioavailability of therapeutic agent(s); (4) enhance the systemic bioavailability by routes of administration currently not practical, (such as oral delivery); and/or (5) modify the pharmacokinetic and pharmacodynamic activity of a therapeutic agent(s).

### BRIEF DESCRIPTION OF THE DRAWINGS

FIG. 1A is a schematic of a solid oral dosage form of a multiparticulate formulation containing therapeutic agent(s), excipients, one or more charge masking agents, and optionally permeation and or dissolution enhancers, encapsulated in a single hard gelatin or cellulose-based capsule or monolithic tablet.

FIG. 1B is a schematic of a solid oral dosage form of a multiparticulate formulation, containing therapeutic agent(s), excipients, one or more charge masking agents, a bioadhesive polymer composition, and optionally permeation and or dissolution enhancers, in a single hard gelatin or cellulose-based capsule, or monolithic tablet, optionally coated with one or more layers of release rate controlling polymers or enteric polymers.

FIG. 1C is a longitudinal section of a longitudinally compressed tablet (“LCT”) containing therapeutic agent(s) excipients, one or more charge masking elements, and optionally permeation and/or dissolution enhancers, disposed in two or three monolithic layers with a slow dissolving or insoluble plug at one end. The LCTs were coated peripherally with a single layer of impermeable PCL film that was heat-sealed to the tablet core. Optionally, bioadhesive polymer layers comprising either anhydride polymers, anhydride oligomers blended with pharmaceutical polymers, catechol-grafted anhydride polymers, or combinations of these polymers can also be applied over the impermeable coating without affecting drug release.

### DETAILED DESCRIPTION OF THE INVENTION

### I. Definitions

As generally used herein “bioadhesives” or “bioadhesive materials” refer to the polymers which are modified to have improved bioadhesion.

“Macromolecule”, as used herein, means a molecule of high relative molecular mass, the structure of which typically consists of multiple repetition of units derived, actually or conceptually, from molecules of low relative molecular mass. Examples include, but are not limited to, polymers, polysaccharides, proteins, peptides, nucleic acids, and lipids. “Macromolecules” include molecules of high relative molecular weight produced by living organisms (i.e., biomolecules) as well as synthetic or semi-synthetic macromolecules. In some embodiments, the macromolecule has a net charge under physiological conditions. Macromolecules generally have a molecular weight of at least 1500, 2000, 2500, 3000, 5000, or 10000 atomic mass units. In contrast, “small molecule” active agents generally have a molecular weight of less than 1000 atomic mass units.

As used herein “bioadhesion” generally refers to the ability of a material to adhere to a biological surface for an extended period of time. Bioadhesion requires a contact between the bioadhesive material and the receptor surface, the bioadhesive material penetrates into the crevice of the surface (e.g. tissue and/or mucus) and chemical bonds form. Thus the amount of bioadhesive force is affected by both the nature of the bioadhesive material, such as a polymer, and the nature of the surrounding medium. Adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups responsible for forming hydrogen bonds are the hydroxyl (—OH) and the carboxylic groups (—COOH). Bioadhesive forces are measured in units of N/m², by methods defined in U.S. Pat. No. 6,197,346 to Mathiowitz et al. Bioadhesive forces, especially those exhibited by tablets, can also be measured in vitro using a Texture Analyser, such as the TA-TX2 Texture Analyser (Stable Micro Systems, Haslemer, Surrey, UK). As described in Michael J. Tobyn et al, Eur. J. Pharm. Biopharm., 41(4):235-241 (1995), a mucoadhesive tablet is attached to a probe on the texture analyzer and lowered until it contacts pig gastric tissue, which is attached to a tissue holder and exposed to liquid at 37° C. to simulate gastric medium. A force is applied for a set period of time and then the probe is lifted at a set rate. Area under the force/distance curve calculations are used to determine the work of adhesion. (See also Michael J. Tobyn et al., Eur. J. Pharm. Biopharm., 42(1):56-61 (1996) and David S. Jones, et al., International J. Pharmaceutics, 151: 223-233 (1997)). Other in vitro techniques include nanoparticle binding percentage (everted sac).

Everted Sac Bioadhesion Assay

The everted sac assay used herein is a modified version of an assay described in the literature. Male, Sprague-Dawley rats weighing 200-250 g are anesthetized with 3% isoflurane prior to a midline abdominal incision. The jejunum is removed, flushed with PBSG, and immediately immersed in fresh PBSG. Segments of jejunum, 6 cm in length, are everted, using a stainless steel rod, and ligated at both ends with silk 0-0 monofilament sutures. The everted sac is filled with approximately 2 ml PBSG and immersed in a nanosphere suspension prepared as follows. Pre-warmed (37° C.) PBSG is added to 60 mg of formulation (0.4% w/v) and bath sonicated for 5 minutes. Once the isolated loop is added to the nanosphere suspensions, samples are placed on an end-over-end mixer at 37° C. for a 30-minute period. During this incubation period nanospheres/microspheres are allowed to adhere spontaneously to the everted intestinal loop. Following incubation, the everted sac is removed, placed in fresh PBS and homogenized with a Cole-Palmer Ultrasonic Homogenizer CV26 with a high gain Q horn and extender set at 40% amplitude for 30 seconds. Homogenized samples are then lyophilized for 48 hours and stored at −18° C. until analysis. The remaining nanosphere/microspheres suspension is centrifuged at 4000 rpm for 5 minutes and remaining supernatant discarded. The formulation pellet is then resuspended in deionized water and centrifuged a final time at 4000 rpm for 5 minutes. Again, the supernatant is discarded. The resulting pellet is lyophilized for 48 hours and stored at −18° C. until analysis. All experiments are completed within 2 hours after jejunum harvesting (n=6).

Techniques for in vitro measurements of bioadhesion include transit/residence time measurements and bioavailability measurements

As used herein, a “charge masking agent” is one which is effective to neutralize, preferably completely, the charge of a therapeutic, prophylactic or diagnostic agent to be delivered, without interfering with its activity.

As used herein “catechol” refers to a compound with a molecular formula of C₆H₆O₂ and the following structure:

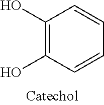

“Microparticle”, as used herein, generally refers to a particle having a diameter of greater than about 10 microns to about 3 mm, preferably from about 10 microns to about 2 mm.

“Nanoparticle”, as used herein, generally refers to a particle having a diameter from about 10 nm to about 10 microns, preferably from 10 nm to about 1 micron, more preferably from about 10 nm to about 0.1 microns. In one embodiment, the particles have a size range from about 500 to about 600 nm. The particles can have any shape but are generally spherical in shape. Nanoparticles having a spherical shape are generally referred to as “nanospheres”. Whole nanospheres may be absorbed into the blood stream and the reduced sizes of the spheres or particles can enhance uptake.

“Bioactive agent” and “active agent” are used interchangeably herein and include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment. Examples include, but are not limited to, small-molecule drugs, peptides, proteins, antibodies, sugars, polysaccharides, nucleotides, oligonucleotides, aptamers, siRNA, nucleic acids, and combinations thereof. “Bioactive agent” includes a single such agent and is also intended to include a plurality of bioactive agents including, for example, combinations of two or more bioactive agents.

“Copolymer” as used herein, generally refers to a single polymeric material that is comprised of two or more different monomers. The copolymer can be of any form, such as random, block, graft, etc. The copolymers can have any end-group, including capped or acid end groups.

“Sufficient” or “effective” as used herein, generally refers to an amount (e.g. mass, volume, dosage, concentration, and/or time period) needed to achieve one or more desired result(s).

“Biocompatible” as used herein, generally refers to a material and any metabolites or degradation products thereof that are generally non-toxic to the recipient and do not cause any significant adverse effects to the subject.

“Biodegradable” as used herein, generally refers to a material that will degrade or erode under physiologic conditions to smaller units or chemical species that are capable of being metabolized, eliminated, or excreted by the subject. The degradation time is a function of polymer composition and morphology. Suitable degradation times are from days to weeks. For example, the polymer may degrade over a time period from seven days to 24 weeks, preferably seven days to twelve weeks, preferably from seven days to six weeks, preferably from seven days to three weeks.

“Molecular weight” as used herein, generally refers to the relative average chain length of the bulk polymer, unless otherwise specified. In practice, molecular weight can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size” as used herein, generally refers to the statistical mean particle size (diameter) of the particles in the composition.

“Controlled release” or “modified release”, as used herein, generally refers to a release profile in which the drug release characteristics of time course and/or location are chosen to accomplish therapeutic or convenience objectives not offered by conventional dosage forms such as solutions, suspensions, or promptly dissolving dosage forms. Delayed release, extended release, and pulsatile release and their combinations are examples of modified release.

As used herein, an “excipient” is an inactive substance other than a chelator or dissolution/stabilization agent, used as a carrier for the insulin or used to aid the process by which a product is manufactured. In such cases, the active substance is dissolved or mixed with an excipient.

As used herein, a “physiological pH” is between 6.8 and 7.6, preferably between 7 and 7.5, most preferably about 7.4.

As used herein, “Cmax” is the maximum or peak concentration of a drug observed after its administration.

As used herein, “Tmax” is the time at which maximum concentration (Cmax) occurs.

### II. Formulations

Formulations containing one or more active agents, one or more bioadhesive elements, one or more charge masking elements, and optionally one or more controlled release (e.g., sustained release, delayed release, or combinations thereof) are described herein. The bioadhesive elements can provide prolonged transit time in the GI tract, intimate contact with absorptive cells, direct transfer of the active agent, increased absorption rate, increased bioavailability, and/or reduced variability in intestinal transit time. The charge masking elements can allow for increased intestinal permeation and promote an increase in bioavailability without chemically altering the active ingredient by masking the charge on the one or more active agents. Increased intestinal absorption and/or increased bioavailability can be evaluated using a variety of techniques in the art including, but not limited to, enzyme-linked immunosorbent assays (ELISA), radioimmuno assays, HPLC, mass spectrometry, HPLC-mass spectrometry, or any other technique specific for the active agent to be measured in blood or another bodily fluid.

A. Active Agents

1. Macromolecules

In some embodiments, the active agent is a macromolecule, such as a polymer, polysaccharide, protein, peptide, nucleic acid, or lipid. The macromolecules can be naturally occurring (i.e., biomolecules) or can be prepared synthetically or semi-synthetically. Macromolecules typically have a high relative molecular weight, e.g., greater than 1500-2000 atomic mass units. Molecular size is an impediment to intercellular (e.g., transcellular) and intracellular (e.g., paracellular) passage of these large molecules. Biomolecules (e.g., macromolecules that are produced by living organisms, such as polysaccharides, proteins, peptides, nucleic acids, and/or lipids) are typically charged at physiological pH. Charge is also an impediment to intercellular and intracellular passage of biomolecules. Charge masking agent, such as those discussed below, can form weak associations with charged active agents, neutralizing the charge on the active agent without altering the chemical structure of the active agent, thus increasing uptake and improving bioavailability.

In one embodiment, the macromolecule is destabilized and/or dissociated into subunits, and a charge masking agent which interacts (e.g., forms hydrogen bonds, electrostatic interactions, etc.) with the charged portions of the molecule to neutralize all or part of the charge. The charge masking agent can enhance absorption through membranes, and provide steric interference, preventing re-aggregation in the bloodstream.

In one embodiment, the macromolecule is heparin. Heparin is a highly-sulfated glycosaminoglycan, widely used as an injectable anticoagulant, and has the highest negative charge density of any known biomolecule. Native heparin is a polymer with a molecular weight ranging from 3 kDa to 40 kDa, although the average molecular weight of most commercial heparin preparations is in the range of 12 kDa to 15 kDa. Heparin is a member of the glycosaminoglycan family of carbohydrates (which includes the closely-related molecule heparin sulfate) and consists of a variably-sulfated repeating disaccharide unit. The most common disaccharide unit is composed of a 2-O-sulfated iduronic acid and 6-O-sulfated, N-sulfated glucosamine, IdoA(2S)-GlcNS(6S). Under physiological conditions, the ester and amide sulfate groups are deprotonated and attract positively-charged counterions to form a heparin salt. It is in this form that heparin is usually administered as an anticoagulant. Heparin can be in the form unfractionated heparin (12-40K Daltons) or fractionated heparins, such as low molecular weight heparin (LMWH, 6K Daltons), and very low molecular weight heparin (VLMWH).

They are several challenges to heparin delivery. For local delivery to the intestines, heparin formulations exhibit significant variability in the residence time in the intestines. Formulations for systemic delivery of heparin often exhibit short small intestine residence times and low intestinal permeability. The use of one or more bioadhesive elements and one or more charge masking element should increase residence time and/or permeability through the intestinal wall. For example, for local delivery of heparin, particles having a larger diameter (e.g., >25 microns) can provide prolonged mucosal attachment in the intestine and provide delivery an effective amount of large hydrophilic macromolecules, such as heparin. For systemic delivery, smaller particles (e.g., ≦1 micron) can provide prolonged mucosal attachment and promote systemic absorption of the hydrophilic biomolecule. Heparin can be in the form of fractionated heparin including all molecular weights, such as heparin (12-40K Daltons), low molecular weight heparin (LMWH, 6K Daltons), and very low molecular weight heparin (VLMWH).

In another embodiment, the macromolecule is Copaxone, also known as glatiramer acetate, copolymer-1 or cop-1. Copaxone is a random polypeptide of alanine, lysine, glutamic acid, and tyrosine, which are the four amino acids found in myelin basic protein. The ratio of alanine to lysine to glutamic acid to tyrosine is ˜5:3:1.5:1. Copaxone has an average molecular weight between 5,000 and 9,000 Daltons.

2. Small Molecule Active Agents

In some embodiments, the active agent is a small molecule active agent. The Biopharmaceutical Classification System (BCS), originally developed by G. Amidon, separates small molecule pharmaceuticals for oral administration into four classes depending on their aqueous solubility and their permeability through the intestinal cell layer. According to the BCS, drug substances are classified as follows:

Class I—High Permeability, High Solubility

Class II—High Permeability, Low Solubility

Class III—Low Permeability, High Solubility

Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development. Class 1 drugs of the BCS system are highly soluble and highly permeable in the gastrointestinal (GI) tract.

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in vivo intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). In the absence of evidence suggesting instability in the gastrointestinal tract, a drug is considered highly soluble when 90% or more of an administered dose, based on a mass determination or in comparison to an intravenous reference dose, is dissolved. A drug substance is considered highly permeable when the extent of absorption in humans is determined to be greater than 90% of an administered dose, based on mass-balance or in comparison to an intravenous reference dose. An IR drug product is considered rapidly dissolving when no less than 85% of the labeled amount of the drug substance dissolves within 30 minuted, using U.S. Pharmacopeia (USP) Apparatus 1 at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

BCS Class I Drugs

Examples of BCS class I drugs include those listed in Kasim et al. Mol. Pharmaceutics 1(1): 85-96 (2004) and Lindenberger et al. Eur. J. Pharm. Biopharm. 58(2):265-78 (2004), such as amitriptyline hydrochloride, biperiden hydrochloride, chloroquine phosphate, chlorpheniramine maleate, chlorpromazine hydrochloride, clomiphene citrate, cloxacillin sodium, ergotamine tartrate, indinavir sulfate, levamisole hydrochloride, levothyroxine sodium, mefloquine hydrochloride, nelfinavir mesylate, neostigmine bromide, phenytoin sodium, prednisolone, promethazine hydrochloride, proguanil hydrochloride, quinine sulfate, salbutamol, warfarin sodium, caffeine, fluvastatin, Metoprolol tartrate, Propranolol, theophylline, verapamil, Diltiazem, Gabapentin, Levodopa, carbidopa, reserpine, ethynyl estradiol, norethindrone, saquinavir mesylate and Divalproex sodium.

Valacyclovir is an antiviral drug which is active against the Herpes viruses. It is used to treat infections with herpes zoster (shingles), herpes simplex genitalis (genital herpes), and herpes labialis (cold sores). Valacyclovir inhibits the replication of viral DNA which is necessary for viruses to reproduce themselves. Valacyclovir is converted to acyclovir in the body.

Gabapentin is a medication indicated as adjunctive therapy in the treatment of partial seizure in epilepsy and for the management of post-herpetic neuralgia (PHN). PHN is the pain that lasts one to three months after shingles has healed. Gabapentin is also used for the treatment of partial seizures in adults and children. Gabapentin is available in capsule, tablet, and oral solution forms. The mechanism of action of gabapentin is unknown, but it has been shown to display analgesic action and anticonvulsant activity. Despite being a Class I drug, gabapentin is not appreciably metabolized in humans. The bioavailability of gabapentin is not dosed proportionally; as the dose increases, the bioavailability of gabapentin decreases. At best, the bioavailability of gabapentin is 60% at a 900 mg dose, given three times a day. Food increases, only slightly, the rate and extent of absorption of gabapentin.

Levodopa is the “gold standard” for the treatment of Parkinson disease. The drug has a narrow absorption window and is absorbed mainly in the proximal small intestine. Gastric emptying of the drug plays an important role in its absorption. There are reports that clearly illustrate that “wearing off” and “on-off” phenomena are associated to the random fluctuation of levodopa levels in the Parkinson patients. Varying gastric emptying results in a considerable inter-subject variability and levels of levodopa need to be monitored to reduce the motor fluctuations.

Many BCS Class I drugs, such as verapamil, levadopa, metformin, and gabapentin, are absorbed only in the upper small intestine and have little or no absorption in the distal small intestine or colon. Many BCS Class I drugs require specific transport carriers in the intestinal tissue for delivery. These carriers can be saturated, thereby preventing absorption of the drug and resulting in sub-optimal absorption.

BCS Class II Drugs

Class II drugs are drugs that are particularly insoluble, or slow to dissolve, but that readily are absorbed from solution by the lining of the stomach and/or the intestine. Hence, prolonged exposure to the lining of the GI tract is required to achieve absorption. Such drugs are found in many therapeutic classes.

Many of the known Class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In a preferred embodiment, the drug is intraconazole and its relatives fluoconazole, terconazole, ketoconazole, and saperconazole. Itraconazole is a Class II medicine used to treat fungal infections and is effective against a broad spectrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it useful for Cushing's syndrome. Itraconazole is available in capsule and oral solution form. For fungal infections the recommended dosage of oral capsules is 200400 mg once a day.

Itraconazole has been available in capsule form since 1992, in oral solution form since 1997, and in an intravenous formulation since 1999. Since itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

A majority of the antimicrobial drugs belong to “Class II” of the Biopharmaceutics Classification System (BCS). Representative antibiotics include amoxicillin, tetracycline, Metronidazole, and clarithromycin.

Other Class II drugs include anti-infective drugs such as sulfasalazine, griseofulvin and related compounds such as griseoverdin; some anti malaria drugs (e.g. Atovaquone); immune system modulators (e.g. cyclosporine); and cardiovascular drugs (e.g. digoxin and spironolactone); and ibuprofen (analgesic); ritonavir, nevirapine, lopinavir (antiviral); clofazinine (leprostatic); diloxanide furoate (anti-amebic); glibenclamide (anti-diabetes); nifedipine (anti-anginal); spironolactone (diuretic); steroidal drugs such as Danazol; carbamazepine, and anti-virals such as acyclovir.

Danazol is derived from ethisterone and is a synthetic steroid. Danazol is designated as 17a-Pregna-2,4-dien-20-yno[2,3-d]-isoxazol-17-ol, has the formula of C₂₂H₂₇NO₂, and a molecular weight of 337.46. Danazol is used in the treatment of endometriosis, fibrocystic breast disease and hereditary angioedema. Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in plasma concentration. Danazol peak concentrations occur within 2 hours, but the therapeutic effect usually does not occur for approximately 6-8 weeks after taking daily doses.

Acyclovir is a synthetic nucleoside analogue that acts as an antiviral agent. Acyclovir is available for oral administration in capsule, tablet, and suspension forms. It is a white, crystalline powder designated as 2-amino-1,9-dihydro-9-[(2-hydroxyethoxy)methyl]-6H-purin-6-one, has an empirical formula of C₈H₁₁N₅O₃ and a molecular weight of 225. Acyclovir has an absolute bioavailability of 20% at a 200 mg dose given every 4 hours, with a half-life of 2.5 to 3.3 hours. The bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue which prevents replication of viral DNA by inhibition and/or inactivation of the viral DNA polymerase, and through termination of the growing viral DNA chain.

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders. Carbamazepine is a white to off-white powder, is designated as 5H-dibenz[b,f]azepine-5-carboxamide, and has a molecular weight of 236.77. It is practically insoluble in water and soluble in alcohol and acetone. The absorption of carbamazepine is relatively slow, despite a bioavailability of 89% for the tablet form. When taken in a single oral dose, the carbamazepine tablets and chewable tablets yield peak plasma concentrations of unchanged carbamazepine within 4 to 24 hours. The therapeutic range for the steady-state plasma concentration of carbamazepine generally lies between 4 and 10 mcg/mL.

BCS Class III and IV Drugs

Class III drugs have good water solubility and poor GI permeability and include proteins, peptides, polysaccharides, nucleic acids, nucleic acid oligomers and viruses. Examples of Class III drugs include abacavir sulfate, amiloride HCl, atropine sulfate, chloramphenicol, folic acid, hydrochlorthazide, lamivudine, methyldopa, mefloquine HCl, penicillamine, pyrazinamide, salbutamol sulfate, valproic acid, stavudine, ethosuximide, ergometrine maleate, colchicines, didanosine, cimetidine, ciprofloxacin, neomycin B, captopril, Atenolol, and Caspofungin.

Caspofungin is a Class III drug and is used to treat serious antifungal agents. Caspofungin acetate is a semisynthetic lipopeptide (echinocendin) compound synthesized from a fermentation product of Glarea lozoyensis. Caspofungin acetate is a hygroscopic, white to off-white powder, which is freely soluble in water and methanol, and slightly soluble in ethanol. The pH of a saturated aqueous solution of caspofungin acetate is approximately 6.6. Caspofungin acetate has an empirical formula of C₅₂H₈₈N₁₀O₁₅.2C₂H₄O₂ and a formula weight of 1213.42. Caspofungin acetate is designated as 1-[(4R,5S)-5-[(2-aminoethyl)amino]-N²-(10,12-dimethyl-1-oxotetradecyl)-4-hydroxy-L-ornithine]-5-[(3IRI)-3-hydroxy-L-ornithine]pneumocandin B₀ diacetate (salt). Caspofungin acts through inhibition of the cell wall synthesis of fungi such as Aspergillus and Candida. Caspofungin acetate is currently available for intravenous injection at 50 mg/day with an elimination half-life of 9-10 hours and is suitable for once-daily regimens. Casposfungin is slowly metabolized by hydrolysis and N-acetylation and also undergoes spontaneous chemical degradation. The bioavailability of Caspofungin is currently 0%.

Class IV drugs are lipophilic drugs with poor GI permeability. Examples include acetazolamide, allopurinol, dapsone, doxycycline, paracetamol, nalidixic acid, clorothiazide, tobramycin, cyclosporin, tacrolimus, and paclitaxel.

Tacrolimus is a macrolide immunosuppressant produced by Streptomyces tsukubaensis. Tacrolimus prolongs the survival of the host and transplanted graft in animal transplant models of liver, kidney, heart, bone marrow, small bowel and pancreas, lung and trachea, skin, cornea, and limb. Tacrolimus acts as an immunosuppressant through inhibition of T-lymphocyte activation through a mechanism that is unknown. Tacrolimus has an empirical formula of C₄₄H₆₉NO₁₂.H₂O and a formula weight of 822.05. Tacrolimus appears as white crystals or crystalline powder. It is practically insoluble in water, freely soluble in ethanol, and very soluble in methanol and chloroform. Tacrolimus is available for oral administration as capsules or as a sterile solution for injection. Absorption of tacrolimus from the gastro-intestinal tract after oral administration is incomplete and variable. The absolute bioavailability of tacrolimus is approximately 17% at a 5 mg dose taken twice a day.

Paclitaxel is a chemotherapeutic agent that displays cytotoxic and antitumor activity. Paclitaxel is a natural product obtained via a semi-synthetic process from Taxus baccata. While having an unambiguous reputation of tremendous therapeutic potential, paclitaxel has some patient-related drawbacks as a therapeutic agent. These partly stem from its extremely low solubility in water, which makes it difficult to provide in suitable dosage form. Because of paclitaxel's poor aqueous solubility, the current approved (U.S. FDA) clinical formulation consists of a 6 mg/ml solution of paclitaxel in 50% polyoxyethylated castor oil (CREMOPHOR EL™) and 50% dehydrated alcohol. Am. J. Hosp. Pharm., 48:1520-24 (1991). In some instances, severe reactions, including hypersensitivity, occur in conjunction with the CREMOPHOR™ administered in conjunction with paclitaxel to compensate for its low water solubility. As a result of the incidence of hypersensitivity reactions to the commercial paclitaxel formulations and the potential for paclitaxel precipitation in the blood, the formulation must be infused over several hours. In addition, patients must be pretreated with steroids and antihistamines prior to the infusion. Paclitaxel is a white to off-white crystalline powder available in a nonaqueous solution for injection. It has an empirical formula of C₄₇H₅₁NO₁₄, and a molecular weight of 853.9. Paclitaxel is highly lipophilic and insoluble in water.

Both Class III and IV drugs are often problematic or unsuitable for sustained release or controlled release. Class III and Class IV drugs are characterized by biomembrane permeability and are commonly delivered parenterally. Traditional approaches to parenteral delivery of poorly soluble drugs include using large volumes of aqueous diluents, solubilizing agents, detergents, non-aqueous solvents, or non-physiological pH solutions. These formulations, however, can increase the systemic toxicity of the drug composition or damage body tissues at the site of administration.

B. Bioadhesive Elements

Bioadhesive elements are included in the formulation to improve gastrointestinal retention via adherence of the formulation to the walls of the GI tract. As used herein “bioadhesive” generally refers to the ability of a material to adhere to a biological surface for an extended period of time. Bioadhesion requires contact between a bioadhesive material and a surface (e.g. tissue and/or cells). Thus the amount of bioadhesive force is affected by both the nature of the bioadhesive material, such as a polymer, and the nature of the surrounding medium. The bioadhesive materials described herein may be used in a wide variety of drug delivery and diagnostic applications. Bioadhesive materials may be formed into microparticles, such as microspheres or microcapsules, or may be a coating on such microparticles. In the preferred embodiment, the material is applied as a coating to any longitudinally compressed tablet.

Bioadhesive polymers are described in U.S. Pat. No. 6,235,313 to Mathiowitz et al. Suitable polymers include polylactic acid (2 kDa MW, types SE and HM), polystyrene, poly(bis carboxy phenoxy propane-co-sebacic anhydride) (20:80) (poly (CCP:SA)), alginate (freshly prepared); and poly(fumaric anhydride-co-sebacic anhydride (20:80) (p[FA:SA]), types A (containing sudan red dye) and B (undyed). Other high-adhesion polymers include p[FA:SA] (50:50) and non-water-soluble polyacrylates and polyacrylamides. In designing bioadhesive polymeric formulations based on polylactides, polymers that have high concentrations of carboxylic acid are preferred. This can be accomplished by using low molecular weight polymers (Mw 2000), since low molecular weight polymers contain high concentration of carboxylic acids at the end groups.

In a preferred embodiment, bioadhesive polymers are typically hydrophobic enough to be non-water-soluble, but contain a sufficient amount of exposed surface carboxyl groups to promote adhesiveness. These include, among others, non-water-soluble polyacrylates and polymethacrylates; polymers of hydroxy acids, such as polylactide and polyglycolide; polyanhydrides; polyorthoesters; blends comprising these polymers; and copolymers comprising the monomers of these polymers. Blending or copolymerization sufficient to provide a certain amount of hydrophilic character can be useful to improve wettability of the materials. For example, about 5% to about 20% of monomers may be hydrophilic monomers. Preferably, the polymers are bioerodable, with preferred molecular weights ranging from 1000 to 50,000 Da, and most preferably 2000 to 20,000 Da.

Polyanhydrides are a preferred type of bioadhesive polymer. The use of certain bioadhesive polymers, particularly polyanhydrides, allows one polymer additive to serve several functions simultaneously to enhance oral uptake. Suitable polyanhydrides include polyadipic anhydride (“p(AA)”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios. p(AA) is a surface-eroding polymer belonging to the polyanhydride family of bioerodable and biocompatible polymers. The polymer is a low molecular weight (2-8 kDa) thermoplastic polymer that quickly degrades to adipic acid monomer and adipic anhydride (both of which are considered GRAS for food applications) over the course of 24 hrs at physiological pH.

Optionally, the polymer is a blend of hydrophilic polymers and bioadhesive hydrophobic polymers. Suitable hydrophilic polymers include hydroxypropylmethylcellulose, hydroxypropylcellulose, carboxymethylcellulose, polyvinylalcohols, polyvinylpyrollidones, and polyethylene glycols. The hydrophobic polymer may contain gastrosoluble polymers that dissolve in stomach contents, such as Eudragit® E100. The hydrophobic polymer may contain entero-soluble materials that dissolve in the intestine above pH 4.5, such as Eudragit® L-100, Eudragit® S-100, cellulose acetate phthalate, hydroxypropyl methylcellulose phthalate, hydroxypropyl methylcellulose acetate succinate, Eastacryl® 30D dispersion from Eastman Chemicals., Sureteric® (polyvinyl acetate phthalate) and Acryl Eze®.

In a preferred embodiment, the bioadhesive polymers contain a water insoluble hydrophobic backbone and nucleophilic functional groups. A compound containing an aromatic group which contains one or more hydroxyl groups, such as catechol, can be grafted onto a polymer or coupled to individual monomers. The polymer or monomer that forms the polymeric backbone may contain accessible functional groups that easily react with molecules contained in the aromatic compounds, such as amines and thiols. In a preferred embodiment, the polymer contains amino reactive moieties, such as aldehydes, ketones, carboxylic acid derivatives, cyclic anhydrides, alkyl halides, acyl azides, isocyanates, isothiocyanates, and succinimidyl esters.

Polymers that contain a catechol functionality are bioadhesive. “Catechol” refers to a compound with a molecular formula of C₆H₆O₂ and the following structure:

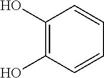

These aromatic groups are substituted for monomers on the backbone of a suitable polymer. The degree of substitution varies based on the desired adhesive strength. It may be as low as 10%, 25%, 50%, or up to 100% substitution. On average, at least 20% of the monomers in a suitable polymeric backbone are substituted with at least one aromatic group.

In a preferred embodiment, the aromatic compound containing one or more hydroxyl groups is catechol or a derivative thereof. Optionally the aromatic compound is a polyhydroxy aromatic compound, such as a trihydroxy aromatic compound (e.g. phloroglucinol, benserazide) or a multihydroxy aromatic compound (e.g. tannin). The catechol derivative may contain a reactive group, such as an amino, thiol, or halide group. The preferred catechol derivative is 3,4-dihydroxyphenylalanine (DOPA), which contains a primary amine. Tyrosine, the immediate precursor of DOPA, which differs only by the absence of one hydroxyl group in the aromatic ring, can also be used. Tyrosine is capable of conversion (e.g. by hydroxylation) to the DOPA form.

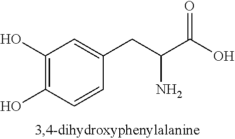 [SMILES: NC(Cc1ccc(O)c(O)c1)C(=O)O]

In another preferred embodiment, the aromatic compound is an amine-containing aromatic compound, such as an amine-containing catechol derivative.

DOPA-containing mucoadhesive polymers include DOPA-maleic anhydride co-polymer, isopthalic anhydride polymer, DOPA-methacrylate polymers, DOPA-cellulosic based polymers, and DOPA-acrylic acid polymers.

Excipients may also be added to improve mucoadhesion. Suitable excipients include FeO/Fe₂O₃, fumaric anhydride oligomer (FAO), L-DOPA-L-DOPA dimer, and adipic anhydride pre-polymer (AAP).

Bioadhesive materials include poly(fumarie acid:sebacic acid) (p[DA:SA]), as described in U.S. Pat. No. 5,955,096 to Mathiowitz et al.; anhydride oligomers, such as Fumaric Anhydride Oligomer and Metal oxides, such as CaO, ferric oxide, magnesium oxide, titanium dioxide, as described in U.S. Pat. No. 5,985,312 to Jacob et al.; and L-DOPA grafted onto butadiene maleic anhydride at approximately 20% substitution efficiency (L-DOPA-BMA)). These bioadhesive materials may be blended with methylmethacrylates, celluloses and substituted celluloses, polyvinylpyrollidones, PEGs, Poly (vinyl alcohols). Alternatively, these materials may be blended with other bioadhesive polymers including p[FA:SA], p(AA), and L-DOPA-BMA.

C. Charge Masking Elements

Bioadhesive formulations have been improved by the addition of one or more charge masking elements or agents. While the charge on a molecule may be manipulated by changing the microenvironment of the molecule (e.g., pH), such techniques are not practical for biomolecules, such as proteins, peptides, and nucleic acids, which are typically charged at physiological pH. Most biomolecules cannot be administered orally due to their large size, susceptibility to acidic hydrolysis and enzymatic degradation, and their highly charged surface. Even when administered parenterally, biomolecules may be absorbed slowly yielding a non-therapeutically effective pharmacokinetic profile.

Many biomolecules are unstable in their active form. In living cells, these molecules are frequently stored as a stable pro-drug, such as pro-hormone, which is enzymatically cleaved at the time of use to produce the active form. Examples of this phenomenon include proinsulin and proparathyroid hormone. Alternatively, the biomolecule(s) may be stored in numerous locations as organized aggregates but in very small quantities at each storage location. As a result, the biomolecule can be released at the same time from multiple sites, resulting in a large concentration gradient at each site and allowing for rapid dissociation and distribution and thus a rapid onset of activity. Insulin is an example of both of these mechanisms. Insulin is cleaved in the secretory granules of beta cells from the proinsulin molecule and stored in each granule as a stable hexameric aggregate. Once released into extracellular fluid, the hexamer experiences a large concentration gradient and rapidly dissociates first to dimers and then to the biologically active monomer which is sufficiently small in molecular size to pass between the tight junctions of cells.

Charge masking agents can provide a variety of functions including, but not limited to, increasing bioavailability of the active agent(s); modifying the pharmacokinetic and pharmacodynamic profile(s) of the active agent(s), e.g. increase or decrease the rate of absorption; increasing the degree of uptake of the active agent(s) into cells; stabilizing a labile active agent or agents, for example, by inhibiting aggregation of the agent or agents; and/or maintaining the active agent or agents in a biologically inactive state in one environment and a biologically active state in another environment.

The charge masking agents(s) are specific to the biomolecule to be delivered, based on at least the chemistry and the charge distribution of the molecule to be delivered. Charge masking agents can mask charge through a variety of different actions. In some embodiments, the charge masking agent masks charge by forming hydrogen bonds with the molecule to be delivered. In other embodiments, the charge masking agent can for electrostatic interactions with the molecules to be delivered. For charge masking agents that can form a single interaction to mask charge, the minimal molar ratio is one to one, although in practice, there is typically an excess of charge masking molecules. In embodiments wherein the charge masking agent can form a plurality of interactions, the minimum ratio is based on the number of interactions. For example, if the molecule to be delivered has 18 charges and the charge masking agents can form two interactions per molecule of charge masking agent, then the minimum ratio is 9:1. The optimal molar ratio can be determined experimentally using a number of methods including, but not limited to, laser light scattering.

Suitable charge masking agents include, but are not limited to, organic acids, such as acetic acid, ascorbic acid, citric acid, glutamic acid, aspartic acid, succinic acid, fumaric acid, maleic acid, and adipic acid and diketopiperazines, monoketopiperazines, piperazines and amino acid such as histadine, arginine, lysine, asparagines, serine, tyrosine, threonine, cystine, glycine and glutamine. Tryptophan, proline, histidine, phenylalanine and tyrosine can be covalently bonded to one or more of a different amino acid or one or more of the same amino acid or any combination thereof to form a molecule that will optimally charge mask the molecule to be delivered (i.e., cargo molecule). In this way, the three dimensional configuration of the charge masking molecule can be tailored to fit the cargo to be masked. The fit can enhance or reduce the strength of the attraction between the charge masking molecule and the cargo. For example, to charge mask heparins, arginine, histidine and lysine are useful as charge masking agents. Alternatively, compounds combining one or more positively charged amino acids with a piperazine or diketopiperazine or monoketopiperazine (e.g. diarginine piperazine) can also be used.

In the embodiments where the active agent is the macromolecule Copaxone, the charge masking agent can be a peptide containing the same peptides as Copaxone, wherein the ratio of lysine and glutamic acid are reversed, i.e., ˜5Ala:1.5Lys:3Glu:1Tyr. At physiological pH, Copaxone should have a net positive charge with an overall hydrophobic nature. Reversing the ratios of lysine and glutamic acid, increases the amount of negative charge on the masking agent which can mask the positive charge of Copaxone. Negatively charged amino acids, such as glutamic acid or aspartic acid, can also be added to mask the charge on Copaxone. Lesser amounts of positively charged amino acids can also be used to mask the negatively charged glutamic acid residues in Copaxone. Other negatively charges species which can be used include, but are not limited to, citrate, acetate, fumarate, and alginate. DOPA has also been shown to bind lysine. Therefore, catechols and polymerized catechols, in particular hydrocaffeic acid, may also be used to charge mask Copaxone.

In another embodiment, Copaxone can be charged masked by the bioadhesive element. In particular embodiments, the bioadhesive element is, or contains, one or more polyanhydrides. The degradation products of polyanhydrides are dicarboxylic acids, such as fumaric acid, adipic acid, sebacic acid, maleic acid, malic acid, phthalic acid, isophthalic acid, aspartic acid, and terephthalic acid, which can mask the positively charged Copaxone.

In one embodiment, the charge masking agent is an organic acid. In another embodiment, the organic acid is a polyacidic acid or polycarboxylic acid. Examples include glucosamine, chitosan, protamine, positively charged amino acids, and combinations of positively charged amino acids.

Acids can be used in the free acid form, as a salt, or a combination of acid and salt. Salts of typical polyacids include sodium acetate, ascorbate, citrate, glutamate, aspartate, succinate, furnarate, maleate, and adipate. Salts of organic acids can be prepared using a variety of bases including, but not limited to, metal hydroxides, metal oxides, metal carbonates and bicarbonates, metal amines, as well as ammonium bases, such as ammonium chloride, ammonium carbonate, etc. Suitable metals include monovalent and polyvalent metal ions. Exemplary metals ions include the Group I metals, such as lithium, sodium, and potassium; Group II metals, such as barium, magnesium, calcium, and strontium; and metalloids such as aluminum. Polyvalent metal ions may be desirable for organic acids containing more than carboxylic acid group since these ions can simultaneously complex to more than one carboxylic acid group.

Charge masking can improve bioavailability and can also modify the pharmacokinetic/pharmacodynamic (PK/PD) profiles for a given active agent, but should not alter the activity of the agent. For example, charge masking can increase the rate of absorption of the active agent into systemic circulation, increasing Maximal blood Concentration (Cmax) and shortening the Time to Maximal blood Concentration (Tmax), which can be advantageous for a variety of active agents. In other examples it can increase the duration of the minimally therapeutically effective concentration (MEC). Charge masking can also increase bioavailability as measured by AUC concentration of the active in the blood. Charge masking is a means of modulating the PK, PD, MEC Cmax, Tmax and AUC of therapeutic agents, thereby making them more safe and effective.

The charge masking agent can be formulated directly with the active agent to be delivered, for example, in microspheres or nanospheres. The charge masking agent can also be incorporated into a matrix containing the active agent. In other embodiments, the charge masking agent can be incorporated into a coating, for example a coating which coats the active agent, such as a controlled release coating, to ensure a high local concentration of the charge masking agent in the vicinity of the drug in vivo during release and/or absorption. The charge masking agent can be added by precipitation with the outer ion of the agent, physical mixing with the dry counter ion so that it will dissolve immediately with the active drug in vivo. In a preferred embodiment, the charge masking agent is formulated so that there is a high local concentration in the vicinity of the agent during release and absorption. The ratio of the counter ion to active drug should be from 1:1 up to 1:100 active drug/charge masking agent. Preferably the charge masking agent is sufficient to make the molecule neutrally charged.

D. Controlled Release Materials

The compositions described here optionally contain one or more controlled release materials. In some embodiment, the controlled release material(s) can be incorporated into nano- or microspheres containing the active agent and the one or more charge masking agents. Alternatively, nano- or microspheres containing the active agent and the one or more charge masking agents can be coated with the controlled release materials.

For drugs requiring absorption in buccal and sublingual regions of the GIT, bioadhesive tablets and particularly bioadhesive multiparticulates and nanoparticles are desirable. Drugs absorbed in these sites avoid first-pass metabolism by liver and degradation by GIT enzymes and harsh pH conditions typically present in the stomach and small intestine. Drugs absorbed in the buccal and sublingual compartments benefit from rapid onset of absorption, typically within minutes of dosing. Particularly suitable are bioadhesive particulates in fast-dissolving dosage forms, e.g., OraSolv (Cima Labs) that disintegrate within 30 sec after dosing and release the bioadhesive particles. Target release profiles include immediate release (IR) and combinations of zero-order controlled release (CR) kinetics and first-order CR kinetics.

For drugs requiring absorption in the stomach and upper small intestine and/or topical delivery to these sites, particularly drugs with narrow absorption windows, bioadhesive, gastroretentive drug delivery systems are the option of choice. Bioadhesive tablets and multiparticulates are formulated to reside for durations greater than 3 hrs and optimally greater than 6 hrs in the fed state. Drug release profiles from these systems are tailored to match the gastric residence times, so that greater than 85% of the encapsulated drug is released during the gastric residence time. Target release profiles include zero-order CR kinetics, first-order CR kinetics and combinations of IR and CR kinetics.

For drugs requiring absorption or topical delivery only in the small intestine, enteric-coated, bioadhesive drug delivery systems are preferred method. Such systems are particularly well suited for topical delivery of therapeutics to Crohn's disease patients. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating.

Following gastric emptying, the enteric coating is “triggered” to dissipate, revealing the underlying bioadhesive coating. Suitable triggers include pH and time duration. Typical of enteric polymers utilizing pH as a trigger are Eudragit polymers manufactured by Rohm America: Eudragit L100-55 dissolves at pH values greater than 5.5, typically found in duodenum; Eudragit L100 dissolves at pH values exceeding 6.0, typically found in jejunum; Eudragit 5100 dissolves at pH values exceeding 7.0, typically found in ileum and the ileocecal junction. Also suitable are cellulosic enteric polymers such as cellulose acetate phthalate.

Time may be used as a trigger to unmask the bioadhesive coating. Coatings that dissolve after 3 hrs when the dosage form is administered in the fed state and after 1-2 hrs when the dosage form is administered in the fasted state are suitable for bioadhesive delivery systems to small intestine. Erosion of soluble polymer layers is one means to achieve a time-triggered, enteric dissolution. Polymers such as HPMC, HPC, PVP, PVA or combinations of the above may be used as time-delayed, enteric coatings and applying thicker coating weights can increase timing of the dissolution of the coating. Suitable enteric coating materials are shown in Table 2.

TABLE 2
Methacrylate-based coating materials

| Functionality | Trade name |
|---|---|
| Anionic polymer of | Eudragit ® L 100-55 - powder, spray dried |
| methacrylic acid and | L 30 D-55 which can be reconstituted for |
| methacrylates with a - | targeted delivery in the duodenum |
| COOH group | Eudragit ® L 30 D-55 - aqueous dispersion, |
| | pH dependent polymer soluble above pH 5.5 |
| | for targeted delivery in the duodenum |
| | Eudragit ® L 100 - powder, pH dependent |
| | polymer soluble above pH 6.0 for targeted |
| | delivery in the jejunum |
| | Eudragit ® S 100 - powder, pH dependent |
| | polymer soluble above pH 7.0 for targeted |
| | delivery in the ileum. |
| | Eudragit ® FS 30 D - aqueous dispersion, |
| | pH dependent polymer soluble above pH 7.0, |
| | requires no plasticizer |
| Cationic polymer with | Eudragit E 100 - granules, pH dependent, |
| a dimethylaminoethyl | soluble in gastric fluid up to 5.0, swellable |
| ammonium group | and permeable above pH 5.0. |
| | Eudragit ® E PO - powder form of E-100 |
| Copolymers of acrylate | Insoluble, High Permeability |
| and methacrylates with | Eudragit ® RL 30D - aqueous dispersion, |
| quarternary ammonium | pH independent polymer for sustained release |
| group. | formulations |
| | Eudragit ® RL PO - powder, pH independent |
| | polymer for matrix formulations |
| | Eudragit ® RL 100 - granules, pH independent |
| | Insoluble, Low Permeability |
| | Eudragit ® RS 30D - aqueous dispersion, |
| | pH independent polymer for sustained release |
| | formulations |
| | Eudragit ® RS PO - powder, pH independent |
| | polymer for matrix formulations |
| | Eudragit ® RS 100 - granules, pH independent |
| Copolymers of acrylate | Eudragit RD 100 - powder, pH independent |
| and methacrylates with | for fast disintegrating films |
| quarternary ammonium | |
| group in combination | |
| with sodium | |
| carboxymethylcellulose | |

Alternately, non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, Triethyl Citrate, Triacetin etc at levels ranging from 0.5 to 50% w/w of the coating and most preferably from 5 to 25% w/w of the coating.

Also suitable are rupturable coating systems, e.g., Pulsincap®, that use osmotic forces of swelling from hydrophilic polymers to rupture enteric membranes to reveal underlying bioadhesive coatings.

Target release profiles include: no more than 10% drug release during the first 3 hrs post-dosing followed by either IR kinetics, zero-order CR kinetics, first-order CR kinetics and combinations of IR and CR kinetics.

For drugs requiring absorption or topical delivery only in the lower small intestine and colon enteric-coated, bioadhesive drug delivery systems are preferred method. Such systems are particularly well suited for topical delivery of therapeutics to patients with Inflammatory Bowel Disease (IBD) including Crohn's disease and Ulcerative Colitis. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating.

Following gastric emptying, the enteric coating is “triggered” to dissipate, revealing the underlying bioadhesive coating. Suitable triggers include pH, time duration and enzymatic action of colonic bacteria. Typical of enteric polymers for delivery to lower GIT utilizing pH as a trigger are Eudragit polymers manufactured by Rohm America: Eudragit S100 and FS dissolves at pH values exceeding 7.0, typically found in ileum and the ileocecal junction.

Time may be used as a trigger to unmask the bioadhesive coating. Coatings that dissolve after 4-5 hrs when the dosage form is administered in the fasted state and after 5-8 hrs when the dosage form is administered in the fed state are suitable for bioadhesive delivery systems to lower small intestine and colon. Erosion of soluble polymer layers is one means to achieve a time-triggered, enteric dissolution. Polymers such as HPMC, HPC, PVP, PVA or combinations of the above may be used as time-delayed, enteric coatings and timing of the dissolution of the coating can be increased by applying thicker coating weights.

Alternately, non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, Triethyl Citrate, Triacetin etc at levels ranging from 0.5 to 50% w/w of the coating and most preferably from 5 to 25% w/w of the coating.

Also, coatings of polymers that are susceptible to enzymatic cleavage by colonic bacteria are another means of ensuring release to distal ileum and ascending colon. Materials such as calcium pectinate can be applied as coatings to tablets and multiparticulates and disintegrate in the lower GIT, due to bacterial action. Calcium pectinate capsules for encapsulation of bioadhesive multiparticulates are also available.

Target release profiles include: no more than 10% drug release during the first 4-5 hrs (fasted state) and 5-8 hrs (fed state) hrs post-dosing followed by either IR kinetics, zero-order CR kinetics, first-order CR kinetics and combinations of IR and CR kinetics.

D. Excipients

The active compounds (or pharmaceutically acceptable salts thereof) may be administered in a formulation wherein the active compound(s) is in an admixture with one or more pharmaceutically acceptable carriers, excipients or diluents. The pharmaceutical formulations may be produced using standard procedures.

The compounds may be complexed with other agents as part of the formulation. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose (HPMC), sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complexes may then be dissolved in an appropriate buffer, for example, phosphate buffered saline or other physiologically compatible solutions. Alternatively, if the resulting complex has poor solubility in aqueous solvents, then it may be formulated with a surfactant such as TWEEN™, or polyethylene glycol, sodium lauryl sulfate, sodium caprate, pluronics, Span 80 and lecithin. Thus, the compounds and their physiologically acceptable solvates may be formulated for administration.

Excipents may also be added to the bioadhesive polymeric composition to alter its porosity and permeability. Suitable excipients may include inorganic and organic materials such as sucrose, hydroxypropyl cellulose, sodium chloride, sodium chloride, xylitol, sorbitol, lactose, dextrose, maltodextrins and dextrates.

Excipents may also be added to the bioadhesive polymeric composition to alter its hydration and disintegration properties. Suitable pH dependent enteric excipients may include cellulose acetate phthalate.

Excipents may also be added as a “wicking agent” to regulate the hydration of the bioadhesive polymeric composition. Suitable excipients may include acdisol, hydroxypropyl cellulose, hydroxypropyl methyl cellulose, cellulose acetate phthalate.

p(AA) prevents coalescence of drug domains within the spray-dried product resulting in increased drug surface area available for dissolution. Additionally, adipic acid monomer generated during polymer degradation increases acidity in the microenvironment of the spray-dried drug particle. By changing the pH, some of the drugs may become more soluble.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxyipropylcellulose (HPC), hydroxpropylmethylcellulose (HPMC), carboxymethylcellulose (CMC) are commonly used for this purpose.

The drugs may optionally be encapsulated or molecularly dispersed in polymers to reduce particle size and increase dissolution. The polymers may include polyesters such as poly (lactic acid) or P[LA], polycaprylactone, polylactide-coglycolide or P[LGA], poly hydroxybutyrate poly β-malic acid); polyanhydrides such as poly (adipic)anhydride or P(AA), poly (fumaric-co-sebacic) anhydride or p[FA:SA], poly (sebacic) anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art. Also suitable are hydrophobic polymers such as polyimides.

The system can also be designed to extend the time period for release by increasing the drug to polymer ratio, with release drawn out to 80% in 90 minutes (in vitro). Increased relative drug concentration is believed to have the effect of increasing the effective drug domain size within the polymer matrix; and increased drug domain size results in slower drug dissolution. In the case of a polymer matrix containing certain types of hydrophobic polymers, the polymer will act as a bioadhesive material and increase the retention time of the drug product in the gastrointestinal tract. Delayed release and extended release compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

As discussed above, examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, methacrylic resins, zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the tablets, multiparticulate formulations, beads, granules, or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet, multiparticulate, bead, or granule remains intact during storage and until administration. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, Pluronics, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine lauryl sulfobetaine, and lecithin.

If desired, the tablets, beads, granules, or particles may also contain minor amounts of nontoxic auxiliary substances, such as wetting or emulsifying agents, dyes, pH buffering agents, or preservatives.

The active agents, alone or in combination with the one or more charge masking elements, may optionally be encapsulated or molecularly dispersed in polymers to reduce particle size. The polymers may include polyesters such as poly (lactic acid) or P[LA], polycaprylactone, poly(lactide-co-glycolide) or P[LGA], polyhydroxybutyrate poly(β-malic acid); polyanhydrides such as poly (adipic)anhydride or P(AA), poly (fumaric-co-sebacic) anhydride or p[FA:SA], poly (sebacic) anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art.

### III. Methods of Making the Formulations

Solid oral dosage forms are typically prepared by blending powdered charged drug or drug particles (i.e. drug in micro or nanoparticles) and the one or more charge masking elements, for example in as a solid precipitation or in solution, with excipients such as those discussed above and compressing the mixture into the form of a tablet. Alternately the mixture may be incorporated into standard pharmaceutical dosage forms such as gelatin capsules and tablets. Gelatin capsules, available in sizes 000, 00, 0, 1, 2, 3, 4, and 5, from manufactures such as Capsugel®, may be filled with mixtures and administered orally. Similarly, macrospheres may be dry blended or wet-granulated with diluents such as microcrystalline cellulose, lactose, cabosil and binders such as hydroxypropylmethylcellulose, hydroxypropylcellulose, carboxymethylcellulose and directly compressed to form tablets. The dimensions of the tablets are limited only by the engineering of dies available for tabletting machines. Dies to form tablets in round, oblong, convex, flat, and bullet designs in sizes ranging from 1 to 20 mm are available. The resulting tablets may weigh from 1 to 5,000 mg and carry microspheres at loadings of 1 to 80% w/w.

The resulting tablets may be coated with sugars, enteric polymers or gelatin to alter dissolution of the tablet. Premature dissolution of the tablet in the mouth may be prevented by coating with hydrophilic polymers, such as hydroxypropylmethylcellulose or gelatin, resulting in dissolution in the stomach.

The tablet or solid oral dosage form may optionally contain absorption enhancers including: sodium caprate, ethylenediamine tetra (acetic acid) (EDTA), Lutrols, polysorbates, sodium lauryl sulfate, citric acid, lauroylcamitine, palmitoylcarnitine, tartaric acid, Vitamin E TPGS (d-alpha-tocopheryl polyethylene glycol 1000 succinate) and other agents known to increase GI permeability by affecting integrity of tight junctions.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The formulation may be in the form of a tablet, capsule, minitab, filled tablet, multiunit filled capsules, multiunits embedded in a rapidly disintegrating tablet, osmotic device, slurry, dispersion, or suspension. In the preferred embodiment, the formulation is a solid oral dosage formulation, such as a tablet, multiparticulate composition, or capsule.

The drug may be incorporated into a polymer matrix by spray drying at any appropriate loading, such as from 1 to 90% w/w, from 1 to 50% w/w, 20 to 70% w/w, from 40 to 60% w/w, and preferably in a range from 20% to 30% w/w. Using other processes such as hot melt extrusion, high shear mixing, the drug loading may vary from 20% to 90% and most preferably from 50-70%.

Drug release rates may be controlled by varying the proportion of drug to carrier in the solution used to prepare the formulation. For example, in some formulations, a drug-polyanhydride system can release drug rapidly, with at least 40% of the drug load in 30 minutes and at least 70% in 60 minutes (in vitro). Drugs are incorporated into the polymer matrix at loadings of 1 to 50% w/w and most preferably in the range of 20-30% w/w.

The system can also be designed to extend the time period for release by increasing the drug to carrier ratio, with release drawn out to 80% in 90 minutes (in vitro). Increased relative drug concentration is believed to have the effect of increasing the effective drug domain size within a polymer matrix; and increased drug domain size results in slower drug dissolution. In the case of a polymer matrix containing certain types of hydrophobic polymers, the polymer will act as a bioadhesive material and increase the retention time of the dosage form in the gastrointestinal tract. Increased drug dissolution rates combined with the bioadhesive properties of the polymer matrix results in (1) increased uptake of the drug and (2) reduction in differences found in the fed and fasted states for BCS Class I drugs.

A. Formation of Drug/Bioadhesive Element/Charge Masking Element Particles

The matrices containing the one or more active agents and one or more bioadhesives elements and/or one or more charge masking agents may be fabricated using any of the encapsulation methods known to those skilled in the art including, but not limited to, solvent evaporation, solvent removal, spray-drying, phase-inversion encapsulation, spontaneous emulsification, coacervation, hot melt encapsulation, pan coating, hot melt extrusion, spray-congealing, fluidized bed coating methods, prilling and grinding. It is understood that the drug-polymer products may be further processed into oral dosage form using any of the standard pharmaceutical techniques including but not limited to tabletting, extrusion-spheronization, hot melt extrusion and fluidized bed coating for multiparticulate dosage forms and capsule-filling.

Because the primary source of adhesiveness and of prevention of aggregation is the nature of the polymer(s) forming the microspheres, the exact method of preparation is critical. The preferred method is spray drying of a solution in which the polymer, the drug, and/or the charge masking agent are dissolved due to its simplicity. Other suitable methods include spray drying of a solution containing dissolved polymer and dispersed fine particles of drug or freeze-drying of a solution containing dissolved polymer, dissolved or suspended drug, and/or dissolved or suspended charge masking agent. Another method involves dissolving a polymer and dissolving or suspending a drug, and then diluting with a large volume (5× to 20×, for example) of a non-solvent for the polymer and the drug, where the solvent is substantially miscible with the non-solvent (at 20×, at least about 8 to 10% soluble). In preferred pairs of solvents and non-solvents, the absolute values of the differences in solubility parameter “delta” between the solvent and the non-solvent is less than about six. (Delta has units of square root of [calories/cm³]).

The resulting particles are suitable for capsules, tableting and other conventional dosage forms.

Spray Drying

In one embodiment, the composition contains a drug/polymer mixture, optionally including the one or more charge masking elements, co-dissolved in a mutual solvent and then spray-dried to form microparticles in the range of 2-100 μm in diameter. Drug loadings can range from 0.5-60% (w/w) drug with polymer, but are typically in the range of about 30% to 40%. Polymer systems contain polymers with bioadhesive qualities, and in the preferred embodiment may include either pure polyanhydride polymers, or mixtures of other biocompatible polymers (e.g., methacrylates, polyesters, polysaccharides) with polyanhydrides. The polymer system acts as a matrix for more rapid dissolution of the drug due to increased surface area by maintaining the micronized drug particle size. Spray dried polymer/drug product is then incorporated with suitable pharmaceutical excipients in a capsule oral dose form or may be filled into a softgelatin capsule after suspending in suitable vehicle.

Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The charge masking agent can also be added to the suspension. The resulting emulsion is stirred until most of the organic solvent is evaporated, leaving solid particles. Several different polymer concentrations can be used, including concentrations ranging from 0.05 to 0.20 g/ml. The solution is loaded with a drug and suspended in 200 ml of vigorously stirred distilled water containing 1% (w/v) poly(vinyl alcohol) (Sigma). After 4 hours of stirring, the organic solvent evaporates from the polymer, and the resulting particles are washed with water and dried overnight in a lyophilizer. Particles with different sizes (1-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

Hot Melt Microencapsulation

Hot melt encapsulation is a thermal processing method in which drug, and optionally the one or more charge masking elements, homogeneously distributed in a polymeric matrix, is forced through a die under controlled conditions. Intense mixing and agitation during processing results in a more uniform dispersion of fine drug particles (Drug Dev. Ind. Pharmacy, Vol 28, issue 7, pp 757, 2003). This method offers the advantages for making spherical pellets, granules, films as well as tablets. The processing includes either a single or twin rotating screw extruder. Depending upon the physical and chemical properties of the drug and other excipients, the drug may be present as undissolved particles, a solid solution or a combination. Plasticizers, anti-oxidants, release controlling agents can be included to improve the processing conditions and stability of the matrix forming bioadhesive polymer. The plasticizers may be solid or liquid in nature.

In one embodiment, the polymer is first melted and then mixed with the solid particles of dye or drug, and optionally the one or more charge masking elements, that have been sieved to less than 50 microns. The mixture is suspended in a non-miscible solvent like silicon oil, and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between one to 1000 microns are obtained with this method. The external surfaces of spheres prepared with this technique are usually smooth and dense. This procedure is used to prepare particles made of polyesters and polyanhydrides. However, this method is limited to polymers with molecular weights between 1000 and 50,000 Da.

Solvent Removal

This technique is primarily designed for polyanhydrides. In this method, the drug, and optionally the one or more charge masking elements, are dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

Extrusion-Spheronization

Core particles may be prepared by the process of granulation-extrusion-spheronization. In this process, micronized drug, optionally containing the one or more charge masking elements, is mixed with microcrystalline cellulose, binders, diluents and water and extruded as a wet mass through a screen. The result is rods with diameters equal to the opening of the extrusion screen, typically in the size range of 0.1 to 5 mm. The rods are then cut into segments of approximately equal length with a rotating blade and transferred to a spheronizer. The spheronizer consists of a rapidly rotating, textured plate which propels rod segments against the stationary walls of the apparatus. Over the course of 1-10 minutes of spheronization, the rods are slowly transformed into spherical shapes by abrasion. The resulting spheroid cores are then discharged from the machine and dried at 40-50° C. for 24-48 hours using tray-driers or fluidized bed dryers. The cores may then be coated with rate-releasing, enteric or bioadhesive polymers using either pan-coating or fluidized-bed coating devices.

B. Preferred Delivery Systems

Tablets, capsules and multi-layer devices can be formulated to produce the desired release and uptake. One can create different release rates for one drug or a combination of drugs by changing the composition of the particulate cores, the relative population of particulate cores containing different drugs or having different formulations, or the type and level of rate controlling polymers as well as the bioadhesive polymer composition coating the particulates. In some embodiment, the delivery systems are in the form of a tablet, such as spherical or elliptical shaped tablet, having a size up to 1 cm, for example from 1 micron up to 1 cm. The tablet can be a single layer tablet or a multilayer tablet, such as a bilayer or trilayer tablet.

Tri-layer tablets provide first-order and, more advantageously, zero-order, release profiles. It is possible to create different release rates for drug by changing the composition of the core matrix, as well as the coating and outer layers.

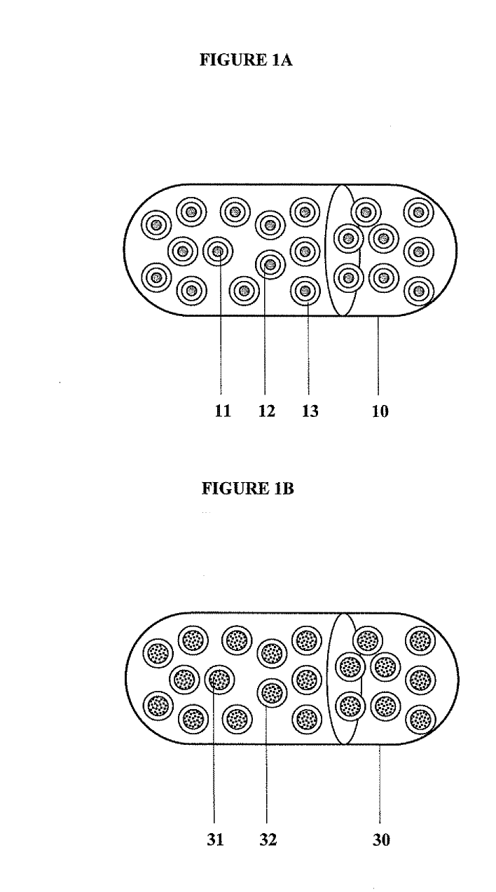

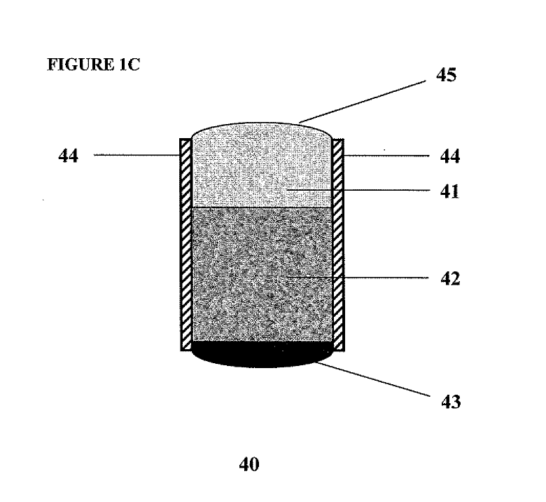

In a preferred embodiment illustrated in FIG. 1A, the solid oral dosage form is a multiparticulate formulation containing drug(s), optionally one or more charge masking elements, excipients, and optionally permeation and or dissolution enhancers, encapsulated in a single hard gelatin or cellulose-based capsule, 10, monolithic matrix. The capsule 10 contains multiparticulates 11 of drug(s), optionally one or more charge masking elements, excipients, and optionally permeation and or dissolution enhancers. The particulates are optionally coated with one or more layers of release rate controlling polymers or enteric polymers 12 and one layer of a bioadhesive polymer composition 13. In embodiments where the multiparticulates of drug do not contain the one or more charge masking elements, the one or more charge masking elements can be incorporated into the coating containing the one or more controlled release elements. The tablet disintegrates quickly in an aqueous medium, releasing its multiparticulate contents.

In another preferred embodiment, illustrated in FIG. 1B, the solid oral dosage form is a multiparticulate formulation, containing drug(s), excipients, a bioadhesive polymer composition, and optionally permeation and or dissolution enhancers, composed in a single hard gelatin or cellulose-based capsule, 30, or monolithic matrix. The capsule contains multiparticulates, 31, of drug(s), excipients, bioadhesive polymer composition, and optionally permeation and or dissolution enhancers. The particulates are optionally coated with one or more layers of release rate controlling polymers or enteric polymers, 32. In another embodiment, the solid oral dosage form is a longitudinally compressed tablet, containing drug, excipients, and dissolution enhancers, composed in a single monolithic layer. The tablet is sealed peripherally with a layer of bioadhesive polymer, leaving the upper and lower sides of the tablet available for drug release.

In another embodiment, the overall shape of the device is designed to be compatible with swallowing. The active agent core can be longitudinally compressed to form a capsule-shaped tablet, which is encapsulated and sealed in a bioadhesive polymeric cylinder. In one embodiment, the core is a multiparticulate containing core, where the active agent is in the form of microparticles. In another embodiment, the active agent core is encapsulated in a bioadhesive polymer cylinder, wherein the tablet is modified to create restricted release openings.

FIG. 1C is a cross-section of a multilayer tablet containing drug in a central matrix of hydrophilic, rate controlling polymers. The inner core is surrounded on two sides by bioadhesive polymer layers, optionally surrounded by an enteric coating. As illustrated in FIG. 1C, the solid oral dosage form is a longitudinally compressed tablet 40 containing one or more drugs, excipients, and optionally permeation and/or dissolution enhancers, disposed in two or more monolithic layers 41. and 42, optionally blocked at one end by a slow-dissolving or non-dissolving passive matrix (also referred to herein as “plug”) 43. The tablet is coated peripherally with a layer of bioadhesive composition 44 leaving the upper side 45 of the tablet available for drug release. First-order and, more advantageously, zero-order release profiles are achievable with this tablet design. The tablet can be designed to provide different immediate release or extended release rates for drugs by changing the composition of the drug layers, or by changing the formulation of the plug. In a preferred embodiment, the solid oral dosage form is a tablet, preferably a trilayer tablet, containing drug in a central matrix of polymer such as hydroxypropylmethylcellulose (“HPMC”) and microcrystalline cellulose (“MCC”) or spray-dried lactose. The inner core is surrounded on two sides by a porous bioadhesive polymer, such as DOPA-BMA polymer or a mixture of bioadhesive p[FA:SA] polymer and Eudragit RS PO. Optionally, the tablet is coated with an enteric coating.

In another embodiment, multiple drug layers are separated by a separating layer and sealed in a bioadhesive polymer cylinder. The resulting capsule can be modified to create restricted release openings. Osmotic systems can be prepared by coating an active agent core containing the one or more charge masking elements with a semi-permeable coating and sealing the coated tablet in a bioadhesive polymer cylinder. In another embodiment, the solid oral dosage form is a longitudinally compressed tablet containing drug, excipients, and dissolution enhancers, composed in two or three monolithic layers, which are separated by slow dissolving passive matrices(also referred to herein as “plugs”). The tablet is coated entirely with a moisture-protective polymer, and then sealed peripherally with a layer of bioadhesive polymer, leaving the upper side, of the tablet available for drug release. The tablet can be designed to provide different immediate release or extended release rates for drugs in a two-pulse or three-pulse fashion by changing the composition or configuration of the drug layers, or by changing the formulation or configuration of the plugs.

In another embodiment, the drug is delivered from an osmotic delivery system. The tablet is coated with a semipermeable membrane. One or both sides of the tablet may be perforated, such as by using a micro-drill or a laser beam to make a micrometer-sized orifice. The tablet is sealed peripherally with a matrix of bioadhesive polymer, leaving the orifice and upper and/or lower sides, of the tablet available for drug release. The semipermeable membrane allows permeation of water into the matrix, leading to the dissolution of drug and creation of osmotic pressure. The increase of osmotic pressure pushes the drug out of the device through the one or more orifice(s) and membrane at controlled rates. Zero-order release profiles are achievable with this tablet design. In another embodiment, the osmotic delivery system is of the “push-pull” design and contains a micronized drug and osmotic agents to draw water across a semi-permeable membrane and a swelling polymer to push the drug out of the device at controlled rates. The entire device is coated with bioadhesive polymers or contains polymer in the matrix of the capsule. The tablet contains an orifice through which the drug is delivered.

In yet another embodiment, a longitudinally compressed tablet containing precompressed inserts of drug and excipients and permeation enhancers and excipients is embedded in a matrix of bioadhesive polymer. Drug is released only at the edge of the tablet and the kinetics of drug release is controlled by the geometry of the inserts.

In one embodiment, the extruded bioadhesive polymer cylinder is prepared via hot-melt extrusion process, where the desired bioadhesive polymer is fed into the extruder as a pellet, flake, powder, etc. along with plasticizer. The materials are blended as they are propelled continuously along a screw through regions of high temperature and pressure to form the polymer extrudate. The extrudate is pushed from the extruder through a die having the desired shape and dimension to form a cylinder. The cylinder is cooled after extrusion. The dimensions of the cylinder can be varied to accommodate the inner core system. The inner diameter of the cylinder can be configured to conform to the desired circumferential dimension of the preformed, pre-pressed inner system containing the therapeutic agent (s). The thickness of the cylinder is determined in part by the polymer/plasticizer type as well its behavior with respect to the external fluid. The bioadhesive nature of the polymer cylinder may also be controlled by mixing different type of polymers and excipients. Inorganic metal oxides may be added to improve the adherence. Pore formers may also be added to control its porosity. Drugs may also be added into the polymer cylinder either as a plasticizer or pore-forming agent. Once formed, the inner system preferably in the form of longitudinally compressed tablet is inserted into the cylinder and two components are fused together to get a finished dosage form.

Extrusion Method for Production of the Hollow Bioadhesive Cylinder

Prior to hot-melt extrusion of the hollow cylinder, the polyanhydride polymer i.e. poly (fumaric-co-sebacic) acid or poly adipic acid and 20% triethyl citrate (based on polymer weight) are mixed in a planetary mixer. Extrusion is performed using either a MP 19 TC25 laboratory scale co-rotating twin screw extruded of APV Baker (Newcastle-under-Lyme, UK) or a Killion extruder (Killian extruder Inc., Cedar Grove, N.J.). Both machines are equipped with a standard screw profile with two mixing sections, an annual die with metal insert for the production of the cylinder and twin screw powder feeder. Typical extrusion conditions are: a screw speed of 5 rpm, a powder feed rate of 0.14 kg/hr and a temperature profile of 125-115-105-80-65° C. from the powder feeder towards the die. The cylinders (internal diameter of 7 mm and wall thickness of 1 mm) are cut into 1 cm long cylinders.

Compression Method for Production of the Hollow Bioadhesive Cylinder

The bioadhesive polymer cylinder may also be formed by a compression process, where the desired bioadhesive polymeric blend is fed into a die of the tabletting machine and compressed using the upper punch attached with a telescopic rod. The telescopic rod pushes the blend and compresses the cylinder. The diameter of the telescopic rod controls the thickness of the bioadhesive cylinder.

Method for Production of the Inner Core System

Inner longitudinally core tablets containing the therapeutic agent and other components are compressed onto a single or multilayer tableting machine equipped with deep fill or regular tooling. For example, the therapeutic agent either alone or in combination with a rate controlling polymer and other excipients is mixed by stirring, ball milling, roll milling or calendaring and pressed into a solid having dimensions conforming to an internal compartment defined by the extruded polymer cylinder. One or more layers containing different therapeutic agents can be included as a multilayer tablet. The inner core system may be a pre-fabricated osmotic system which is inserted into the bioadhesive cylinder with orifices aligned along the open ends of the cylinder.

Method of Insertion of the Inner Core System into the Bioadhesive Cylinder

The preformed inner core with a diameter slightly smaller than the inner diameter of the cylinder is either manually or mechanically inserted into the cylinder and heated to fuse the two units. Alternately, the core insertion into the cylinder may also be done by a positive placement core insertion mechanism on the tableting machine. Initially, the extruded cylinder may be placed into the die of the machine followed by insertion of the compressed core into the internal compartment of the cylinder and the two components compressed to get the finished dosage form. Alternatively, the dosage form is prepared via simultaneous extrusion of the bioadhesive cylinder and expandable inner composition using an extruder capable of such an operation. Alternatively, the dosage form is prepared via compression coating process. The preformed inner core with length similar to the diameter of the die is mechanically inserted over a bed of bioadhesive polymer/excipients blend by a positive core insertion mechanism. After core insertion, additional bioadhesive polymer/excipients blend is added over the core and compressed to get the final dosage form.

### IV. Methods of Use

The compositions described herein can be used for local administration or systemic administration of one or more active agents, particularly charged active agents, such as protein, peptides, and nucleic acids. The compositions described herein can be used to delivery an effective amount of an active agent to one or more locations along the gastrointestinal tract.

The Gastrointestinal Tract

In a normal human adult male, the GI tract is approximately 25 feet long and consists of the following components: 1) mouth (buccal cavity; includes salivary glands, mucosa, teeth and tongue); 2) pharynx; 3) esophagus and cardia; 4) stomach, which includes the antrum and pylorus; 5) intestine, including the small intestine, which has three parts-duodenum, jejunum, and ileum, and the large intestine, which also has three parts-cecum, colon (ascending colon, transverse colon, descending colon and sigmoid flexure) and rectum; and 6) the anus.

Under normal circumstances, a drug may be expected to remain in the stomach for 2 to 4 hours (gastric emptying time) and in the small intestine for 4 to 10 hours, although there is a substantial variation between people, and even in the same person on different occasions. The gastric emptying time for a dosage form is most rapid with a fasting stomach, becoming slower as the food content is increased. Changes in gastric emptying time and/or intestinal motility can affect dosage form transit time and thus the opportunity for drug dissolution and absorption. (Ansel et al. Pharmaceutical Dosage Forms and Drug Delivery Systems 6th ed. Williams and Wilkins, 1995). Generally drugs are better absorbed in the small intestine (because of the larger surface area) than in the stomach, therefore quicker stomach emptying will increase drug absorption. For example, a good correlation has been found between stomach emptying time and peak plasma concentration for acetaminophen. The quicker the stomach emptying (shorter stomach emptying time) the higher the plasma concentration. Also slower stomach emptying can cause increased degradation of drugs in the stomach's lower pH; e.g. proton pump inhibitors, carbidopa. Food can affect the rate of gastric emptying. For example fatty food can slow gastric emptying and retard drug absorption. Generally the extent of absorption is not greatly reduced. Occasionally absorption may be improved. Griseofulvin absorption is improved by the presence of fatty food. Apparently the poorly soluble griseofulvin is dissolved in the fat and then more readily absorbed.

The various gastrointestinal regions and typical transit times are shown in the following Table 1.

TABLE 1
Characteristics of Gastro-intestinal Physiology

| | | | Blood | Surface | Transit | By- |
|---|---|---|---|---|---|---|
| REGION | pH | Membrane | Supply | Area | Time | pass liver |
| BUCCAL | approx 6 | thin | Good, fast | small | Short unless | yes |
| | | | absorption | | controlled | |
| | | | with low dose | | | |
| ESOPHAGUS | 6 | Very | — | small | short | — |
| | | thick, no | | | | |
| | | absorption | | | | |
| STOMACH | 1-3 | normal | good | small | 30-40 minutes, | no |
| | decomposition, | | | | reduced | |
| | weak acid | | | | absorption | |
| | unionized | | | | | |
| DUODENUM | 4-5.5 | normal | good | very large | very short | no |
| | bile duct, | | | | (6″ long), | |
| | surfactant | | | | window effect | |
| | properties | | | | | |
| SMALL | 6-7 | normal | good | very large | about 3 hours | no |
| INTESTINE | | | | 10-14 ft, | | |
| | | | | 80 cm²/cm | | |
| LARGE | 6.8-7 | — | good | not | long, up | lower colon, |
| INTESTINE | | | | very large | to 24 hr | rectum yes |
| | | | | 4-5 ft | | |

A coordinated combination of controlled release, bioadhesive elements, and charges masking agents can be used to achieve release in the desired region where enhanced uptake occurs due to the inclusion of the bioadhesive elements. The charge masking agents can also improve uptake of the active agent and thus increase bioavailability.

In one embodiment, the compositions described herein are formulated for local administration of heparin, such as low molecular weight heparin (LMWH) to the GI tract to treat Crohn's disease, Irritable Bowel Syndrome, and/or colitis. The composition can be in the form nanospheres containing heparin and the charge masking agent, encapsulated in bioadhesives microspheres, which are in turn encapsulated in a hard or soft capsule, such as a gelatin capsule. The capsule can be coated with an enteric coating to prevent degradation of the capsule in the stomach. Alternatively, the capsule itself can contain an enteric polymer which avoids the need for a polymer coating.

In another embodiment, the compositions described herein are formulated for systemic administration of LMWH via the oral route. In this embodiment, heparin can be formulated in a bioadhesive microsphere or nanosphere containing one or more charge masking elements and encapsulated in a hard or soft capsule, such as a gelatin capsule. The capsule can be coated with a pH-sensitive polymer, such as a EUDRAGIT®. Formulations for systemic delivery of heparin can provide pain free delivery, reduce peak-to-trough effects, improve patient compliance, be used to treat larger and/or more diverse patient populations, and/or be used to treated new indications.

In still another embodiment, the compositions described herein are formulated for systemic administration of Copaxone. Copaxone is an immunomodulator drug currently used to treat multiple sclerosis. It is a random polymer of four amino acids found in myelin basic protein, namely glutamic acid, lysine, alanine, and tyrosine, and may work as a decoy for the immune system. Although the clinical definition of multiple sclerosis requires two or more episodes of symptoms and signs, Copaxone is approved for treatment after single episodes. It is also used to treat relapsing-remitting multiple sclerosis. It is currently administered by subcutaneous injection.

1. A formulation comprising
a charged therapeutic, prophylactic or diagnostic active agent to be delivered,
one or more bioadhesive elements, and
one or more charge masking elements sufficient to neutralize the charge on the agent to be delivered.

2. The formulation of claim 1, further comprising one or more controlled release materials selected from sustained or extended release materials, delayed release materials, and combinations thereof.

3. (canceled)

4. The formulation of claim 1, wherein the active agent is in a bioadhesive polymeric matrix or bioadhesive coated matrix, wherein the bioadhesive polymer comprises a water insoluble hydrophobic backbone and mucophilic functional groups.

5. The formulation of claim 1, wherein the active agent is in the form of particles or granules.

6. The formulation of claim 1, further comprising a permeation or absorption enhancer.

7. The formulation of claim 6, wherein the enhancer is selected from the group consisting of sodium caprate, ethylenediamine tetra(acetic acid) (EDTA), citric acid, lauroylcarnitine, palmitoylcarnitine, tartaric acid, Vitamin E TPGS, and other agents that increase gastrointestinal permeability.

8. The formulation of claim 1, wherein the bioadhesive element is a water-insoluble hydrophobic polymer selected from the group consisting of polyanhydrides, poly(meth)acrylate, polyhydroxy acids, polyesters, and copolymers thereof.

9. The formulation of claim 1, wherein the bioadhesive element comprises a polymer backbone substituted with one or more catechols.

10. The formulation of claim 9, wherein the catechol is 3,4-dihydroxyphenylalanine (DOPA).

11. The formulation of claim 9, wherein the polymeric backbone is a hydrophobic polymer selected from the group consisting of polyanhydrides, polyacrylates, polyorthoesters, polyesters, and polyhydroxy acids.

12. (canceled)

13. The formulation of claim 1, wherein the bioadhesive element comprises anhydride oligomers.

14. The formulation of claim 1, wherein the bioadhesive element comprises a metal oxide.

15-17. (canceled)

18. The formulation of claim 1, wherein the charge masking element is a polyacidic agent or polycarboxylic acid or salt thereof.

19. The formulation of claim 18 wherein the polyacidic agent is an acid or salt selected from the group consisting of acetic acid, ascorbic acid, citric acid, glutamic acid, aspartic acid, succinic acid, fumaric acid, maleic acid, and adipic acid.

20. The formulation of claim 18 wherein the salts are prepared using a base selected from the group consisting of metal hydroxides, metal oxides, metal carbonates and bicarbonates, metal amines, and ammonium bases.

21. The formulation of claim 18 wherein the charge masking agent is a complex of a polyvalent metal ion and a polyacid containing more than one carboxylic acid group.

22. The formulation of claim 1, wherein Cmax is different than a reference formulation consisting of the active agent in the absence of the charge masking element(s), the bioadhesive element(s), or combinations thereof.

23. The formulation of claim 22 having greater efficacy of uptake as measured by area under the curve for the plasma concentration over time than a reference formulation consisting of the active ingredient in the absence of the charge masking element(s), the bioadhesive element(s), or combinations thereof.

24. The formulation of claim 1, wherein the formulation is a solid oral dosage formulation selected from the group consisting of tablets, capsules, minitabs, filled tablets, and osmotic tablets.

25. The formulation of claim 1, wherein the therapeutic agent is a heparin.

26-28. (canceled)

29. The formulation of claim 1, wherein the therapeutic agent is copaxone.

30. A method of delivering an active agent to a patient in need thereof, comprising administering to the patient a formulation comprising
a charged therapeutic, prophylactic or diagnostic active agent to be delivered,
one or more bioadhesive elements, and
one or more charge masking elements sufficient to neutralize the charge on the agent to be delivered.

31. The method of claim 30, wherein following administration, the formulation releases the active agent into the buccal/sublingual area.

32. The method of claim 30, wherein following administration, the formulation releases the active agent in the stomach.

33. The method of claim 30, wherein following administration, the formulation releases the active agent in the colon.
